(19) United States
(12) Patent Application Publication
(10) Pub. No.: US 2026/0036561 A1
(43) Pub. Date: Feb. 5, 2026

(54) Recording System and Method for the Automated Documentation of Defects in Semi-finished Products

(71) Applicant: Bayerische Motoren Werke Aktiengesellschaft, Muenchen (DE)

(72) Inventors: Ingo HEINLE, Inzell (DE); Clemens SCHUBERT, Leipzing (DE)

(21) Appl. No.: 19/100,802

(22) PCT Filed: Sep. 27, 2023
(86) PCT No.: PCT/EP2023/076702

§ 371 Date: Feb. 3, 2025

(30) Foreign Application Priority Data
Oct. 12, 2022 (DE) 10 2022 126 564.1

(51) Int. Cl. G01N 33/00

(52) U.S. Cl. CPC G01N 33/0078

(57) ABSTRACT

A recording system for the automated documentation of defects in semi-finished products, includes at least one measuring device configured to record at least one measured value of a semi-finished product that is processed in the manufacturing plant, and a data processing device, which is connected to the at least one measuring device and which receives the at least one measured value. The data processing device includes: a database in which database entries for documenting defects in the semi-finished product can be saved; a comparison device, which is configured to compare the at least one measured value with predefined range limits and to save the at least one measured value lying outside the range limits in a database entry; and a damage recording device configured to record data on the extent of the damage caused by the defect if the at least one measured value is outside the range limits.

### BACKGROUND AND SUMMARY

The present disclosure relates to a recording system for the automated documentation of defects in semifinished products. Furthermore, the present disclosure relates to a method for the automated documentation of defects in semifinished products.

German patent application DE 199 21 748 A1 describes a system and a method for controlling automated and personnel-free logistics, rental, and sales systems, which provides the possibility of a complaint with suppliers.

It is an object of the present disclosure to provide a recording system and a method which simplify the documentation of defects in a semifinished product.

The stated object is achieved by a recording system for the automated documentation of defects in semifinished products. The recording system comprises at least one measuring device, which is arranged on or in a manufacturing facility and is configured to record at least one measured value of a semifinished product processed in the manufacturing facility, wherein the at least one measured value characterizes properties of the semifinished product. In addition, the recording system comprises a server-based or cloud-based data processing unit, which has a first interface via which it is connected to the at least one measuring device and receives the at least one measured value from the at least one measuring device. The data processing unit comprises a database, in which database entries for the documentation of defects in the semifinished product are storable, a comparison unit configured to compare the at least one measured value to predetermined range limits of a permissibility range and, for the case that the at least one measured value lies outside the range limits, to store the at least one measured value in a database entry created for the documentation of the defect, and a damage recording unit configured, for the case that the at least one measured value lies outside the range limits, to record data on the scope of the damage caused by the defect and store these data in the database entry created for the documentation of the defect.

The recording system according to the present disclosure enables a defect in a semifinished product to be documented automatically. If the comparison of measured values of the semifinished product to predetermined range limits has the result that the properties of the semifinished product lie outside the range limits, a new database entry is automatically created in which the items of information on this defect in the semifinished product are brought together. The measured values of the defective semifinished product are recorded together with items of information on the scope of the damage caused by the defect in the database entry. The items of information brought together in the database entry can be used as the basis for the preparation of a complaint directed to the supplier. An advantage of such a recording system is that the data required for a complaint can be established with little additional work effort so that the preparation of complaints is facilitated and reduced in cost. A further advantage is that due to the accurate recording of the scope of damage, a prioritization of the individual damage cases in accordance with their economic relevance is enabled.

The present disclosure additionally relates to a method for the automated documentation of defects in semifinished products in a manufacturing facility by a server-based or cloud-based data processing unit, which comprises a database in which database entries for the documentation of defects in the semifinished product are storable, wherein the data processing unit is connected via a first interface to at least one measuring device attached on or in the manufacturing facility. The method comprises steps of recording at least one measured value which characterizes properties of the semifinished product by the at least one measuring device, transmitting the at least one measured value from the at least one measuring device to the data processing unit, and comparing the at least one measured value to predetermined range limits of a permissibility range. In addition, for the case that the at least one measured value lies outside the range limits, the method comprises storing the at least one measured value in a database entry created for the documentation of the defect, and recording data on the scope of the damage caused by the defect and storing these data in the database entry created for the documentation of the defect.

Advantageous embodiments and refinements which can be used individually or in combination with one another are the subject matter of the claims and the following description.

According to one advantageous embodiment, the damage recording unit is configured, for the case that the at least one measured value lies outside the range limits, to record which section of the semifinished product processed in the manufacturing facility is affected by the defect and to store this information in the database entry created for the documentation of the defect.

Furthermore, it is advantageous if the damage recording unit is configured, for the case that the at least one measured value lies outside the range limits, to record how large the section of the semifinished product processed in the manufacturing facility is that is affected by the defect, and to store this information in the database entry created for the documentation of the defect.

The recording system preferably comprises a position recording unit, which is arranged on or in the manufacturing facility and is configured to record a position of the semifinished product processed in the manufacturing facility, wherein the data processing unit is configured, for the case that the at least one measured value lies outside the range limits, to retrieve the position of the semifinished product from the position recording unit once or repeatedly and store it in the database entry created for the documentation of the defect.

Preferably, the damage recording unit is configured, for the case that the at least one measured value lies outside the range limits, to record which processing steps have already been carried out using the semifinished product affected by the defect and to store this information in the database entry created for the documentation of the defect.

It is advantageous if the damage recording unit is configured to ascertain a damage sum proceeding from the data on the scope of the damage caused by the defect and to store the damage sum in the database entry created for the documentation of the defect.

The database entry stored in the database preferably contains the at least one measured value recorded by the at least one measuring device, the data on the scope of the damage caused by the defect, and a damage sum.

The data processing unit is preferably configured to prioritize the database entries stored in the database in accordance with the amount of the damage sum.

According to one preferred embodiment, the data processing unit is configured, for the case that the at least one measured value lies outside the range limits, to automatically request a laboratory test for the semifinished product affected by the defect.

The data processing unit preferably has a second interface and is connected via the second interface to a quality management system and is configured to transmit at least one documentation of a defect stored in the database to the quality management system via the second interface.

It is advantageous if the data processing unit and the quality management system are configured to automatically prepare a complaint on the basis of the documentation of a defect stored in the database.

Preferably, the comparison unit is configured to record a change trend of the measured values recorded by the at least one measuring device and to output a prior warning even before a range limit is exceeded or fallen below.

The data processing unit is preferably configured to derive the range limits of the permissibility range for measured values recorded by a measuring device from predetermined specification boundaries of a specification of the semifinished product in consideration of the measurement inaccuracy of the respective measuring device.

The data processing unit is preferably configured to derive the range limits of the permissibility range for measured values recorded by a measuring device, in that a tolerance band, which reflects the measurement inaccuracy of the respective measuring device, is added in each case to predetermined specification boundaries of a specification of the semifinished product.

Furthermore, the data processing unit is preferably configured to determine, on the basis of a set of measured values recorded in succession by a measuring device, a scatter band of this set of measured values and to determine the tolerance band proceeding from the scatter band by statistical methods.

Furthermore, the data processing unit is preferably configured to determine, on the basis of a set of measured values recorded in succession by a measuring device, the interquartile range of the set of measured values and to convert the interquartile range by statistical methods into a tolerance band which reflects the measurement inaccuracy of the respective measuring device.

According to one preferred embodiment, the semifinished product is sheet metal and the manufacturing facility is a manufacturing facility of vehicle construction. Preferably, the manufacturing facility is one of the following: a plate trimming facility, a forming unit, a pressing line.

The at least one measuring device arranged in or on the manufacturing facility of vehicle construction is preferably configured to record at least one measured value of the semifinished product which characterizes at least one of the following properties of the sheet metal: thickness of the sheet metal, tribological properties such as lubricant quantity and/or roughness of the sheet metal, elastoplastic properties of the sheet metal, number of weld seams of the sheet metal, chemical properties of the sheet metal.

It is advantageous if the at least one measuring device arranged in or on the manufacturing facility of vehicle construction is at least one of the following: a measuring device for optically recording the sheet metal thickness, a measuring device for optically recording weld seams, a measuring device for inductively recording weld seams, a measuring device for optically recording a lubricant quantity on the sheet metal, an eddy current sensor for recording elastoplastic properties of the sheet metal.

Further advantageous embodiments will be described in more detail hereinafter on the basis of an embodiment illustrated in the drawings, to which the present disclosure is not restricted, however.

### BRIEF DESCRIPTION OF THE DRAWINGS

FIG. 1 shows a recording system for the automated documentation of defects in semifinished products.

FIG. 2 shows the structure of a database entry in the database of the recording system.

FIG. 3 shows the sequence of the method according to the present disclosure for the automated documentation of defects in semifinished products.

### DETAILED DESCRIPTION OF THE DRAWINGS

In the following description of preferred embodiments of the present disclosure, identical reference signs designate identical or comparable components.

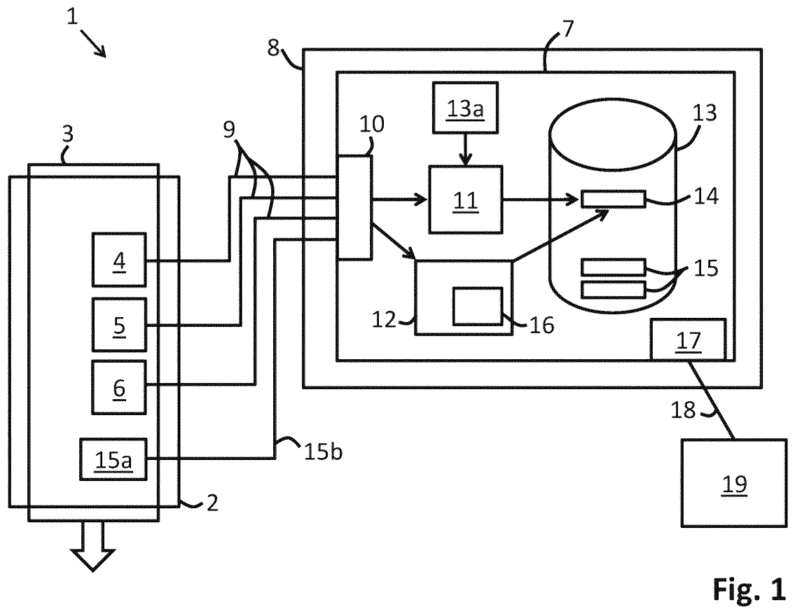

FIG. 1 shows a recording system 1 for recording defects in a semifinished product. Semifinished products such as sheet metal are generally acquired from various suppliers and processed in the manufacturing. To enable correct further processing, the properties of these semifinished products have to meet predetermined specifications. If the properties of a semifinished product move outside the previously defined specification, this defective semifinished product cannot be further processed. In this case, this semifinished product is complained about to the supplier, who thereupon has to reimburse for the resulting damages. The damage caused by a defective semifinished product also comprises the entire expenditure which has occurred during the processing of the defective semifinished product. The documentation and the assertion of such damages are simplified using the recording system 1 described hereinafter.

FIG. 1 shows a manufacturing facility 2 by way of example, which is configured for processing a semifinished product 3. The manufacturing facility 2 can be, for example, a manufacturing facility of vehicle construction, for example, a plate trimming facility, a forming unit, or a pressing line.

Measuring devices for recording properties of the semifinished product 3 are arranged on or in the manufacturing facility 2. In the example shown in FIG. 1, a first measuring device 4, a second measuring device 5, and a third measuring device 6, which determine different properties of the processed semifinished product 3, are arranged on or in the manufacturing facility 2. The measured values recorded by the measuring devices 4, 5, 6 are transmitted to a data processing unit 7, which is installed on a server 8. For this purpose, the first measuring device 4, the second measuring device 5, and the third measuring device 6 are connected via first data connections 9 to a first interface 10 of the data processing unit 7.

The data processing unit 7 comprises a comparison unit 11. a damage recording unit 12, and a database 13 for the documentation of defects. In the comparison unit 11, the measured values recorded by the measuring devices 4, 5, 6 are compared to predetermined range limits 13a of a permissibility range. These range limits 13a for the measured values recorded by a measuring device can be derived, for example, from the specification boundaries of the specification of the semifinished product in consideration of the measurement inaccuracy of the respective measuring device. For example, the range limits 13a can be determined in that a tolerance band, which represents the measurement inaccuracy of the respective measuring device, is added in each case to the specification boundaries of the semifinished product specification. The tolerance band can be determined by statistical methods, for example. For example, on the basis of a sequence of measured values recorded in succession by a measuring device, a scatter band of this sequence of measured values can be determined. Proceeding from this scatter band, a tolerance band which reflects the measurement inaccuracy of the measuring device can then be determined by statistical methods. The specifications of various semifinished products can be stored, for example, in a specification database.

If the comparison performed by the comparison device 11 has the result that all measured values are within the range limits 13a, the semifinished product 3 corresponds to the specifications and there is no reason for a complaint. In contrast, if at least one of the recorded measured values is outside the predetermined range limits 13a, the semifinished product 3 does not correspond to the specifications. In this case, a defect of the semifinished product 3 for which a complaint can be made exists. For the case that such an infringement of the specification is detected, it can be provided that the data processing unit 7 outputs a warning message. In addition, it can be provided, for example, that the data processing unit 7 automatically sends an email message to responsible employees.

For the documentation of the defect, the comparison unit 11 generates a new database entry 14 in the database 13, in which older database entries 15 on defects that have occurred earlier are already stored. In the new database entry 14, the comparison unit 11 initially stores an identifier of the process. This identifier can comprise, for example, one or more of the following: a serial number of the work piece, an item number of the work piece, an item number of the semifinished product, an identifier of the manufacturing facility. In addition, the measured values of the semifinished product, which partially do not correspond to the required specifications, recorded by the measuring devices 4, 5, 6 are stored in the new database entry 14 in order to document the defect.

In addition to the recorded measured values, the extent of the damage arising due to the defect of the semifinished product 3 is also documented. In order to determine the extent of the damage. the damage recording unit 12 ascertains which section of the semifinished product 3 is affected by the defect and how large this section affected by the defect is. If the semifinished product 3 is sheet metal, for example, it is recorded how many meters of the sheet metal unrolled from the coil are defective. For this purpose, a position recording unit 15a can be provided on the part of the manufacturing facility 2, which is connected via a second data connection 15b to the first interface 10 of the data processing unit 7 and is configured to transmit the respective strip position of the semifinished product to the data processing unit 7. It can then be determined from the strip positions at the beginning and at the end of the specification infringement, for example, how large the section of the semifinished product affected by the defect is. For example, it can be determined how many meters of the sheet metal unrolled from the coil do not correspond to the specification.

In addition, it can be provided that the time is established at the beginning and at the end of the specification infringement and upon resumption of regular operation. Machine downtimes can be derived from these time marks, for example, which are also part of quantifiable damage.

In addition, it can be provided that the damage recording unit 12 determines which processing steps have already been performed for the defective semifinished product 3 and which superfluous expenditure has been caused by these processing steps. The expenditure caused by the defective semifinished product 3 is refundable and can be invoiced to the supplier.

In addition, it can be provided that the processing results achieved using the defective semifinished product are documented. If cracks and wrinkles occur during the forming of the semifinished product which does not conform to specifications, for example, such information can also be used for the documentation of the extent of the damage.

The recorded data on the scope of the damage caused by the defect are stored in the new database entry 14. In addition, it can be provided that the comparison unit 11 automatically orders a laboratory test for the semifinished product 3 affected by the defect upon occurrence of such a defect.

The damage recording unit 12 can additionally comprise a damage assessment unit 16, which ascertains a damage sum on the basis of the recorded data on the scope of the damage caused by the defect. This damage sum is also stored in the new database entry 14. The damage thus documented can then be sorted and prioritized by the data processing unit 7 in accordance with the damage sum.

The data processing unit 7 can additionally be connected via a second interface 17 and a third data connection 18 to a quality management system 19. The quality management system 19 can access the data processing unit 7 via the second interface 17 and read database entries 14. 15 from the database 13. On the basis of the documentation of a defect stored in a database entry, the quality management system 19 can prepare a complaint to the supplier in a completely or partially automated manner based on these data.

In the solution shown in FIG. 1, the data processing unit 7 and the database 13 are implemented on a server 8. Alternatively or additionally, it could also be provided that the data processing unit and the database are outsourced into a cloud.

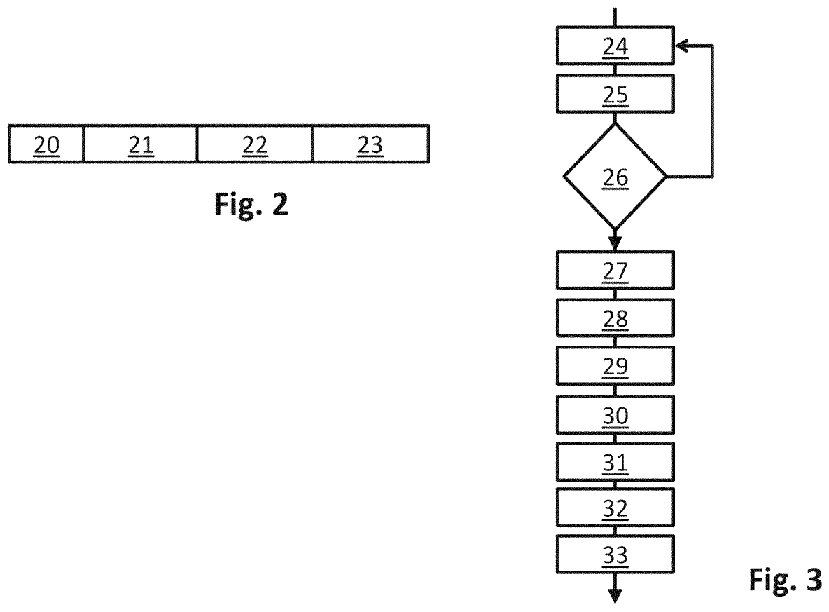

FIG. 2 shows the structure of a database entry 14 of the database 13. An identifier of the procedure is stored in the first region 20 of the database entry 14. This identifier can comprise, for example, one or more of the following: a serial number of the work piece, an item number of the work piece, an item number of the semifinished product 3, an identifier of the manufacturing facility. The measured values recorded by the measuring devices 4, 5, 6 on the properties of the semifinished product 3, which at least partially lie outside the predetermined range limits, are stored in the second region 21 of the database entry. Items of information on the scope of the damage are stored in the third region 22 of the database entry, for example, items of information about which section of the semifinished product 3 is affected by the defect, how large this section is, how long the machine downtime was, and which processing steps had already been carried out using the defective semifinished product 3. Finally, a damage sum ascertained by the damage assessment unit 16 is stored in the fourth region 23 of the database entry 14. These items of information stored in the database entry 14 enable the quality management system 19 to prepare a complaint directed to the supplier in a completely or partially automated manner.

In the example shown in FIG. 1, the properties of the processed sheet metal recorded by the measuring devices 4, 5, 6 can be, for example, at least one of the following: the sheet metal thickness, the tribological properties of the sheet metal, such as the amount of lubricant located on the sheet metal or the roughness of the sheet metal, the elastoplastic properties of the sheet metal, the number of weld seams of the sheet metal, the chemical properties of the sheet metal. Suitable measuring devices can be provided for recording one or more of these properties, preferably measuring devices that operate contactlessly. The sheet metal thickness can be determined, for example, with the aid of two lasers directed onto the upper and lower side of the sheet metal. An oil layer adhering to the surface of the sheet metal can also be measured using optical measuring methods. An eddy current sensor can be used to record the elastoplastic properties of the sheet metal. The weld seams, which have to be identified by the supplier by separate markings, can be recorded and counted by detecting these markings, for example, by a light barrier. Alternatively or additionally, the weld seams can be detected by an inductive measuring method.

FIG. 3 schematically shows the sequence of the method according to the present disclosure for the automated documentation of defects in a semifinished product 3. In first step 24, the measured values are recorded by the measuring devices 4, 5, 6 attached on or in the manufacturing facility and transmitted to the data processing unit 7. In second step 25, these measured values are compared to the predetermined range limits 13a. In third step 26, it is queried whether the measured values lie within the range limits 13a. If this is the case, there is no reason for a complaint and the method jumps back to first step 24.

However, if at least one measured value lies outside the range limits 13a, in fourth step 27, a new database entry 14 is created for the documentation of the defect. In addition, it can be provided that a laboratory test is automatically requested. In fifth step 28, an identifier of the procedure is stored in the new database entry 14. Moreover, the recorded measured values are stored in the new database entry 14.

Sixth, seventh, and eighth steps 29, 30, 31 are used to document the scope of the damage caused by the defect. In sixth step 29, it is determined how large the section of the semifinished product 3 affected by the defect is. In seventh step 30, further data on the scope of the damage caused by the defective semifinished product are recorded. These can be, for example, data on machine downtimes, on the processing steps performed using the defective semifinished product 3, and/or on the processing results achieved using the defective semifinished product 3. In eighth step 31, the damage is assessed and a damage sum is ascertained.

In ninth step 32, the database entries 14, 15 stored in the database 13 are sorted or prioritized in accordance with the level of the damage sum. In tenth step 33, the data contained in the database entries 14, 15 are transmitted to the quality management system 19, which automatically generates one or more complaints therefrom.

The features disclosed in the preceding description, the claims, and the drawings can be important for the implementation of the present disclosure in its various embodiments both individually and in any combination.

### LIST OF REFERENCE SIGNS

1 recording system
2 manufacturing facility
3 semifinished product
4 first measuring device
5 second measuring device
6 third measuring device
7 data processing unit
8 server
9 first data connections
10 first interface
11 comparison unit
12 damage recording unit
13 database
13a range limits
14 new database entry
15 older database entries
15a position recording unit
15b second data connection
16 damage assessment unit
17 second interface
18 third data connection
19 quality management system
20 first region of the database entry
21 second region of the database entry
22 third region of the database entry
23 fourth region of the database entry
24 first step
25 second step
26 third step
27 fourth step
28 fifth step
29 sixth step
30 seventh step
31 eighth step
32 ninth step
33 tenth step

1.-11. (canceled)

12. A recording system for an automatic documentation of defects in semifinished products, comprising:
at least one measuring device, which is arranged on or in a manufacturing facility and is configured to record at least one measured value of a semifinished product processed in the manufacturing facility, wherein the at least one measured value characterizes properties of the semifinished product,
a server-based or cloud-based data processing unit, which has a first interface via which it is connected to the at least one measuring device and receives the at least one measured value from the at least one measuring device, wherein the data processing unit has:
a database, in which database entries for a documentation of defects in the semifinished product are storable,
a comparison unit, which is configured to compare the at least one measured value to predetermined range limits of a permissibility range and, for a case that the at least one measured value lies outside the predetermined range limits, to store the at least one measured value in a database entry created for the documentation of a defect,
a damage recording unit, which is configured, for the case that the at least one measured value lies outside the predetermined range limits, to record data on a scope of a damage caused by the defect and store the data in the database entry created for the documentation of the defect.

13. The recording system according to claim 12, wherein the damage recording unit is configured, for the case that the at least one measured value lies outside the predetermined range limits, to record which section of the semifinished product processed in the manufacturing facility is affected by the defect and to store this information in the database entry created for the documentation of the defect.

14. The recording system according to claim 12, wherein the damage recording unit is configured, for the case that the at least one measured value lies outside the predetermined range limits, to record how large the section of the semifinished product processed in the manufacturing facility is that is affected by the defect, and to store this information in the database entry created for the documentation of the defect.

15. The recording system according to claim 12, wherein the damage recording unit is configured, for the case that the at least one measured value lies outside the predetermined range limits, to record which processing steps have already been carried out using the semifinished product affected by the defect and to store this information in the database entry created for the documentation of the defect.

16. The recording system according to claim 12, wherein the damage recording unit is configured to ascertain a damage sum proceeding from the data on the scope of the damage caused by the defect and to store the damage sum in the database entry created for the documentation of the defect.

17. The recording system according to claim 16, wherein the data processing unit is configured to prioritize the database entries stored in the database in accordance with an amount of the damage sum.

18. The recording system according to claim 12, wherein the database entry stored in the database contains the at least one measured value recorded by the at least one measuring device, the data on the scope of the damage caused by the defect, and a damage sum.

19. The recording system according to claim 12, wherein the data processing unit has a second interface and is connected via the second interface to a quality management system and is configured to transmit at least one documentation of a defect stored in the database to the quality management system via the second interface.

20. The recording system according to claim 12, wherein the data processing unit is configured to derive the predetermined range limits of the permissibility range for measured values recorded by the at least one measuring device, and wherein a tolerance band, which reflects a measurement inaccuracy of a respective measuring device, is added in each case to predetermined specification boundaries of a specification of the semifinished product.

21. The recording system according to claim 20, wherein the data processing unit is configured to determine, on a basis of a set of measured values recorded in succession by the at least one measuring device, a scatter band of the set of measured values and to determine the tolerance band proceeding from the scatter band by statistical methods.

22. A method for an automated documentation of defects in semifinished products in a manufacturing facility by a server-based or cloud-based data processing unit, which comprises a database, in which database entries for a documentation of defects in a semifinished product are storable, wherein the data processing unit is connected via a first interface to at least one measuring device attached on or in the manufacturing facility,
the method comprising:
recording at least one measured value, which characterizes properties of the semifinished product, by the at least one measuring device;
transmitting the at least one measured value from the at least one measuring device to the data processing unit;
comparing the at least one measured value to predetermined range limits of a permissibility range;
for a case that the at least one measured value lies outside the predetermined range limits, storing the at least one measured value in a database entry created for a documentation of a defect; and
for the case that the at least one measured value lies outside the predetermined range limits, recording data on a scope of a damage caused by the defect and storing the data in the database entry created for the documentation of the defect.

23. The method according to claim 22. further comprising for the case that the at least one measured value lies outside the predetermined range limits, recording which section of the semifinished product processed in the manufacturing facility is affected by the defect and to store this information in the database entry created for the documentation of the defect.

24. The method according to claim 22, further comprising for the case that the at least one measured value lies outside the predetermined range limits, recording how large the section of the semifinished product processed in the manufacturing facility is that is affected by the defect, and storing this information in the database entry created for the documentation of the defect.

25. The method according to claim 22, further comprising for the case that the at least one measured value lies outside the predetermined range limits, recording which processing steps have already been carried out using the semifinished product affected by the defect and storing this information in the database entry created for the documentation of the defect.

26. The method according to claim 22, further comprising ascertaining a damage sum proceeding from the data on the scope of the damage caused by the defect and storing the damage sum in the database entry created for the documentation of the defect.

27. The method according to claim 26, further comprising prioritizing the database entries stored in the database in accordance with an amount of the damage sum.

28. The method according to claim 22, wherein the database entry stored in the database contains the at least one measured value recorded by the at least one measuring device, the data on the scope of the damage caused by the defect, and a damage sum.

29. The method according to claim 22, wherein the data processing unit has a second interface and is connected via the second interface to a quality management system, and the method further comprising transmitting at least one documentation of a defect stored in the database to the quality management system via the second interface.

30. The method according to claim 22, further comprising deriving the predetermined range limits of the permissibility range for measured values recorded by the at least one measuring device, and adding a tolerance band, which reflects a measurement inaccuracy of a respective measuring device, in each case to predetermined specification boundaries of a specification of the semifinished product.

31. The method according to claim 30, further comprising determining, on a basis of a set of measured values recorded in succession by the at least one measuring device, a scatter band of the set of measured values, and determining the tolerance band proceeding from the scatter band by statistical methods.
